(19) United States
(12) Patent Application Publication
(10) Pub. No.: US 2021/0110883 A1
(43) Pub. Date: Apr. 15, 2021

(54) A METHOD AND APPARATUS FOR BUILDING A PEDIGREE FOR A SPECIFIC DISEASE BASED ON A FAMILY TREE

(71) Applicant: KONINKLIJKE PHILIPS N.V., EINDHOVEN (NL)

(72) Inventors: Jin Qu, Shanghai (CN); Fubiao Xia, Shanghai (CN); Alexander Ryan Mankovich, Somerville, MA (US); Raymond J. Krasinski, Suffern, NY (US)

(21) Appl. No.: 17/256,806

(22) PCT Filed: Jun. 24, 2019
(86) PCT No.: PCT/EP2019/066597

§ 371 Date: Dec. 29, 2020

(30) Foreign Application Priority Data
Jun. 29, 2018 (CN) PCT/CN2018/093826

(51) Int. Cl. G16B 10/00; G16H 10/60; G16H 50/70; G16H 50/30; G06F 21/62; G06F 16/22

(52) U.S. Cl. CPC G16B 10/00; G16H 10/60; G06F 16/2255; G16H 50/30; G06F 21/6245; G16H 50/70

(57) ABSTRACT

Various embodiments relate to a method and apparatus for building a pedigree for a disease based on a family tree, the method comprising the steps of receiving, by a pedigree service, a family health condition request, transmitting, by the pedigree service, a request for the family tree, transmitting, by the pedigree service, a data request for health conditions of relatives on the family tree to an access control system, verifying, by the access control system, consent of the relatives on the family tree, receiving, by the pedigree service, health conditions of the relatives from a health data storage and drawing, by the pedigree service, a pedigree for the disease based on the health conditions of the relatives.

### TECHNICAL FIELD

This disclosure relates generally to building a pedigree, and more specifically, but not exclusively, to building a pedigree for a specific disease based on a family tree.

### BACKGROUND

Genetic diseases are any disease that is caused by an abnormality in an individual's genome. The abnormality in the genome may range from a discrete mutation in a single base of a single gene to a chromosome abnormality. Genetic diseases may be inherited from the parents, while others may be caused by acquired mutations in a pre-existing gene or group of genes.

For genetic diseases inherited from the parents, these diseases may include single gene inheritance, multifactorial inheritance, chromosome abnormalities, and/or mitochondrial inheritance patterns. The multifactorial inheritance diseases may be caused by a combination of environmental factors and mutations in multiple genes.

Therefore, as members of a family have many factors in common, including their genes, environment, and lifestyle, a family medical history may help identify people in the family with a greater chance of having the genes for common disorders, for example, heart disease, high blood pressure, stroke, certain cancers, and diabetes.

By recognizing patterns of disorders among relatives, healthcare professionals may be able to determine whether an individual in the family, other family members, or future generations may be at an increased risk of developing a particular condition.

As a result, individuals at an increased risk for certain conditions, may receive frequent screening starting at an earlier age.

The individual may also alter his or her lifestyle to lower the risk of developing some diseases, for example, a healthy diet, regular exercise, quitting smoking.

Even before birth, for example, a fertilized egg, may receive genome editing to remove existing defects in the genome of the fertilized egg.

### SUMMARY

A brief summary of various embodiments is presented below. Embodiments address a method and apparatus for building a pedigree for a specific disease based on a family tree.

A brief summary of various example embodiments is presented. Some simplifications and omissions may be made in the following summary, which is intended to highlight and introduce some aspects of the various example embodiments, but not to limit the scope of the invention.

Detailed descriptions of example embodiments adequate to allow those of ordinary skill in the art to make and use the inventive concepts will follow in later sections.

Various embodiments relate to a method for building a pedigree for a disease based on a family tree, the method including the steps of receiving, by a pedigree service, a family health condition request, transmitting, by the pedigree service, a request for the family tree, transmitting, by the pedigree service, a data request for health conditions of relatives on the family tree to an access control system, verifying, by the access control system, consent of the relatives on the family tree, receiving, by the pedigree service, health conditions of the relatives from a health data storage and drawing, by the pedigree service, a pedigree for the disease based on the health conditions of the relatives.

In an embodiment of the present disclosure, the method for building a pedigree for a disease based on a family tree, the method further including transmitting, by the pedigree service, the pedigree to a patient.

In an embodiment of the present disclosure, the method for building a pedigree for a disease based on a family tree, the method further including determining, by the pedigree service, which of the relatives on the family tree are affected by the disease.

In an embodiment of the present disclosure, the method for building a pedigree for a disease based on a family tree, the method further including identifying, by the pedigree service, a pattern of inheritance.

In an embodiment of the present disclosure, the method for building a pedigree for a disease based on a family tree, the method further including identifying, by the pedigree service, a risk for the relatives on the family tree for the disease.

In an embodiment of the present disclosure, the access control system verifies the relatives on the family tree and a signature from a service which provides the family tree.

In an embodiment of the present disclosure, the relatives on the family tree, using a Merkle tree, attach data on a blockchain with a hash value.

In an embodiment of the present disclosure, when the pedigree service requests the health condition data from the access control system, the pedigree service attaches a link to the blockchain.

In an embodiment of the present disclosure, the access control system verifies the blockchain and transmits the health condition data.

In an embodiment of the present disclosure, genome data from the patient is sent to a network which determines whether the genome data belongs to relatives on the network.

Various embodiments relate to a pedigree service for building a pedigree for a disease based on a family tree, the pedigree service including a memory; and a processor configured to receive a family health condition request, transmit a request for the family tree, transmit a data request for health conditions of relatives on the family tree to an access control system, receive verification of consent for the relatives on the family tree, receive health conditions of the relatives from a health data storage and draw a pedigree for the disease based on the health conditions of the relatives.

In an embodiment of the present disclosure, the pedigree service for building a pedigree for a disease based on a family tree, the processor further configured to transmit the pedigree to a patient.

In an embodiment of the present disclosure, the pedigree service for building a pedigree for a disease based on a family tree, the processor further configured to determine which of the relatives on the family tree are affected by the disease.

In an embodiment of the present disclosure, the pedigree service for building a pedigree for a disease based on a family tree, the processor further configured to identify a pattern of inheritance.

In an embodiment of the present disclosure, the pedigree service for building a pedigree for a disease based on a family tree, the processor further configured to identify a risk for the relatives on the family tree for the disease.

In an embodiment of the present disclosure, an access control system verifies the relatives on the family tree and a signature from a service which provides the family tree.

In an embodiment of the present disclosure, the relatives on the family tree, using a Merkle tree, attach data on a blockchain with a hash value.

In an embodiment of the present disclosure, when the pedigree service requests the health condition data from the access control system, the pedigree service attaches a link to the blockchain.

In an embodiment of the present disclosure, the access control system verifies the blockchain and transmits the health condition data.

In an embodiment of the present disclosure, genome data from the patient is sent to a network which determines whether the genome data belongs to relatives on the network.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate example embodiments of concepts found in the claims and explain various principles and advantages of those embodiments.

These and other more detailed and specific features are more fully disclosed in the following specification, reference being had to the accompanying drawings, in which:

FIG. 1 illustrates a block diagram of the system architecture for building a pedigree for a specific disease using a family tree of the current embodiment;

FIG. 2 illustrates a family tree for a specific disease of the current embodiment; and

FIG. 3 illustrates a block diagram of a real-time data processing system of the current embodiment.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments. Descriptors such as “first,” “second,” “third,” etc., are not meant to limit the order of elements discussed, are used to distinguish one element from the next, and are generally interchangeable.

Sharing health data and records among immediate family members is simple compared to sharing health data and records for more than two generations in a family which may become difficult due to the coordination and availability of the health data and records.

However, even though it is difficult, for genetic disease or rare disease, the doctor of a patient requires access to this health data and records for other immediate family members of the patient, but also requires access to the health data and records of the patient's extended relatives including and potentially beyond three generations. The required health data and records may be for relatives unknown to the patient. However, this relative's health data and records may assist the doctor to correctly diagnosis and prepare a prognosis for the patient.

The current embodiment is directed towards automatically and effectively retrieving consent to access the health data and records and/or genome data of the patient's family members and distant relatives to ensure the accuracy and efficiency of retrieval of this information for the doctor to correctly diagnose the patient.

The current embodiment is a method and system for building a pedigree for a specific disease for a family member based on a family tree and/or a genome data comparison result.

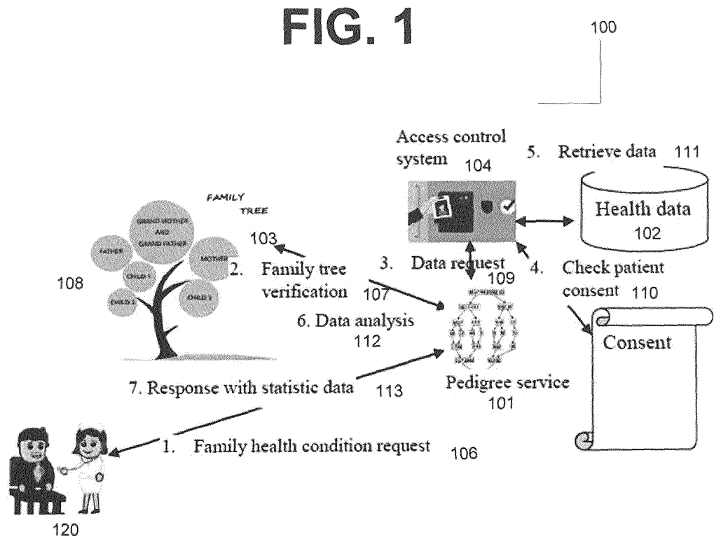

FIG. 1 illustrates a block diagram of the system 100 architecture for building a pedigree for a specific disease using a family tree of the current embodiment.

The system 100 includes a pedigree service 101, a health data database 102, and a family tree service 103. Once a patient 120 initializes a family health condition request to the pedigree service 101 for a specific disease or a group of suspected diseases, the pedigree service 101 transmits a request to the family tree service 103 to identify relatives on the patient's family tree 108.

The pedigree service 101 may send a data request to the access control system 104 of the health data storage 102 to request health data and records of the relatives on the patient's family tree 108. The health data database 102 may respond to the pedigree service 101 with the requested data. Finally, the pedigree service 101 may output a pedigree for the patient's requested disease and return the result to the patient 120.

For a specific disease, after the pedigree service 101 receives the health data and records for the relatives on the family tree 108, the service outputs a pedigree for the patient's requested disease.

The pedigree which is output from the pedigree service organizes information about the medical history of the relatives, states who may be affected, identifies the pattern of inheritance and calculates who may be at risk for the patient's requested disease.

The process begins with step 106 where the patient 120 initializes a family health condition request to the pedigree service 101 for a specific disease or a group of suspected diseases. The process proceeds to step 107 where, after receiving the request from the patient 120 at step 106, the pedigree service 101 transmits a request to the family tree service 103 to identify relatives on the patient's family tree 108.

The process then proceeds to step 109 where the pedigree service 101 transmits a data request to the access control system 104 of the health data storage 102 to request health data and records of the relatives on the patient's family tree 108. The process then proceeds to step 110 where the access control system 104 verifies the consent of the relatives.

If consent is granted, the access control system 104 can verify that the consent policy permits the relative's health data and records to be shared for building a specific disease pedigree, the process proceeds to step 111 where the relevant health data and records are shared with the access control system 104 and the pedigree service 101. If consent is not granted, the access control system 104 cannot verify that the consent policy permits the relative's health data and records to be shared for building a specific disease pedigree, the process ends and the health data and records are not shared.

The process then proceeds to step 112, which uses the health records and data from the patient's relatives, and performs data analysis and draws a pedigree for the patient's requested disease. Then the process proceeds to step 113 which returns the pedigree for the patient's requested disease to the patient 120.

For a specific disease, after the pedigree service 101 receives the health data and records from relatives on the patient's family tree 106, the pedigree service 101 draws a pedigree for the patient's requested specific disease. The pedigree organizes information about the medical history of the relatives by illustrating which relatives are affected by the specific disease, identifies the pattern of inheritance and calculates who is at risk for the patient's requested disease.

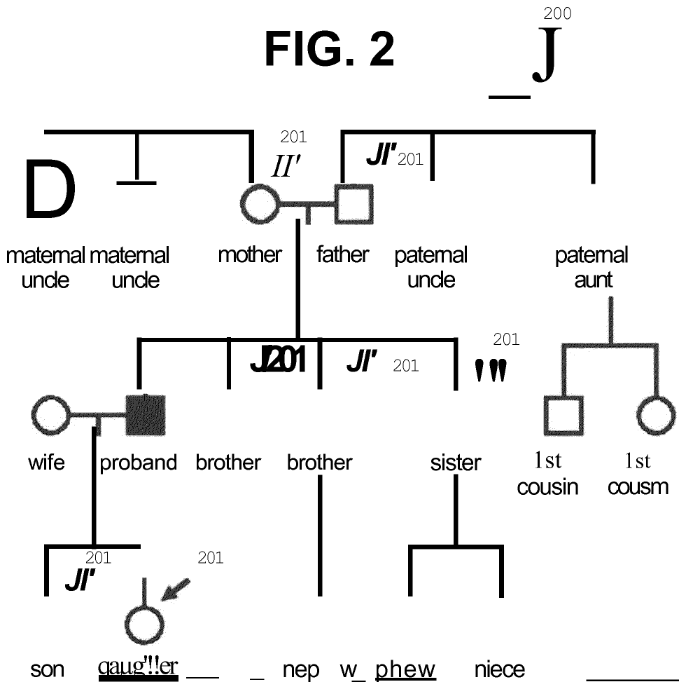

FIG. 2 illustrates a family tree 200 for a specific disease of the current embodiment.

The relatives marked with an arrow 201 have the requested disease condition.

A family tree may be provided by a third party service, for example, Ancestry.com, Facebook, etc.

If the pedigree service is requested by the patient to provide a pedigree for a specific disease, the pedigree service generates a request on behalf of the patient to a family tree generator service for a list of the patient's family members on their family tree. The family tree service provides a list of family members to the pedigree service with a signature.

When the pedigree service requests from the access control system, the health data and records for a condition for the specific disease and the list of family members is provided by the family tree. The access control system verifies the family members list and the signature.

When the family tree service and the family member list are verified, the access control system may respond to the pedigree service with the requested health condition for the family member list.

In another embodiment, a blockchain may be used to provide a family tree service. For example, a Merkle tree which may be built on a blockchain. In the blockchain, each family member may verify the data on the block and may attach their data to the blockchain with a hash value.

Therefore, when the pedigree service requests health data and records for the patient's family from the access control system, the pedigree service may attach the link of the family tree blockchain to the access control system. The identity of the relatives on the patient's family tree may be stored in a hash value and therefore may remain anonymous to the access control system for confidentiality purposes. The access control system may verify the Merkle tree of the blockchain and respond to the pedigree service with the health data and records.

In another embodiment, for some distant relatives, identities may not appear in the patient's family tree. However, for specific rare diseases, it may be necessary to be aware of the data and records of these relatives.

The Beacon Network may provide family tree information for distant relatives. For example, the patient may provide genome data on their mitochondrial DNA or Y chromosome to the Beacon Network. The Beacon Network may compare the patient's genome data on their mitochondrial DNA or Y chromosome with that of other users on the Beacon Network to identify whether any data belongs to any other users which may assist in identifying these relatives. If the Beacon Network identifies relatives of the patient, the Beacon Network may return the family list to the pedigree service. The Beacon Network may maintain anonymity of the relatives of the patient, by only providing relevant genetic data and not the identification information for the patient's relative.

In another embodiment, the system may be used to identify potential genetic disease risks for a fertilized egg and mark the relevant genes.

For example, if parents are aware that there may be inherited diseases in their family, the parents can initialize a pedigree search for the inherited diseases. The pedigree service may provide a pedigree of requested diseases. The physician of the parents may perform genome editing on the fertilized egg of the parents according to pedigree and the genome data of the fertilized egg.

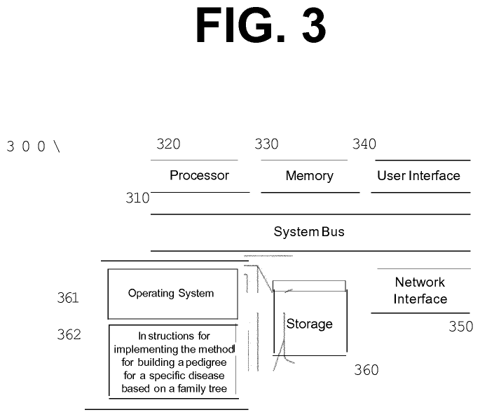

FIG. 3 illustrates an exemplary hardware diagram 300 for implementing a method for building a pedigree for a specific disease based on a family tree. As shown, the device 300 includes a processor 320, memory 330, user interface 340, network interface 350, and storage 360 interconnected via one or more system buses 310. It will be understood that FIG. 3 constitutes, in some respects, an abstraction and that the actual organization of the components of the device 300 may be more complex than illustrated.

The processor 320 may be any hardware device capable of executing instructions stored in memory 330 or storage 360 or otherwise processing data. As such, the processor may include a microprocessor, field programmable gate array (FPGA), application-specific integrated circuit (ASIC), or other similar devices.

The memory 330 may include various memories such as, for example L1, L2, or L3 cache or system memory. As such, the memory 330 may include static random access memory (SRAM), dynamic RAM (DRAM), flash memory, read only memory (ROM), or other similar memory devices.

The user interface 340 may include one or more devices for enabling communication with a user such as an administrator. For example, the user interface 340 may include a display, a mouse, and a keyboard for receiving user commands. In some embodiments, the user interface 340 may include a command line interface or graphical user interface that may be presented to a remote terminal via the network interface 350.

The network interface 350 may include one or more devices for enabling communication with other hardware devices. For example, the network interface 350 may include a network interface card (NIC) configured to communicate according to the Ethernet protocol. Additionally, the network interface 350 may implement a TCP/IP stack for communication according to the TCP/IP protocols. Various alternative or additional hardware or configurations for the network interface 350 will be apparent.

The storage 360 may include one or more machine-readable storage media such as read-only memory (ROM), random-access memory (RAM), magnetic disk storage media, optical storage media, flash-memory devices, or similar storage media. In various embodiments, the storage 360 may store instructions for execution by the processor 320 or data upon with the processor 320 may operate. For example, the storage 360 may store a base operating system 361 for controlling various basic operations of the hardware 300 and instructions for implementing the method for building a pedigree for a specific disease based on a family tree 362.

It will be apparent that various information described as stored in the storage 360 may be additionally or alternatively stored in the memory 330. In this respect, the memory 330 may also be considered to constitute a “storage device” and the storage 360 may be considered a “memory.” Various other arrangements will be apparent. Further, the memory 330 and storage 360 may both be considered “non-transitory machine-readable media.” As used herein, the term “non-transitory” will be understood to exclude transitory signals but to include all forms of storage, including both volatile and non-volatile memories.

While the host device 300 is shown as including one of each described component, the various components may be duplicated in various embodiments. For example, the processor 320 may include multiple microprocessors that are configured to independently execute the methods described herein or are configured to perform steps or subroutines of the methods described herein such that the multiple processors cooperate to achieve the functionality described herein. Further, where the device 300 is implemented in a cloud computing system, the various hardware components may belong to separate physical systems. For example, the processor 320 may include a first processor in a first server and a second processor in a second server.

It should be apparent from the foregoing description that various exemplary embodiments of the invention may be implemented in hardware. Furthermore, various exemplary embodiments may be implemented as instructions stored on a non-transitory machine-readable storage medium, such as a volatile or non-volatile memory, which may be read and executed by at least one processor to perform the operations described in detail herein. A non-transitory machine-readable storage medium may include any mechanism for storing information in a form readable by a machine, such as a personal or laptop computer, a server, or other computing device. Thus, a non-transitory machine-readable storage medium may include read-only memory (ROM), random-access memory (RAM), magnetic disk storage media, optical storage media, flash-memory devices, and similar storage media and excludes transitory signals.

It should be appreciated by those skilled in the art that any blocks and block diagrams herein represent conceptual views of illustrative circuitry embodying the principles of the invention. Implementation of particular blocks can vary while they can be implemented in the hardware or software domain without limiting the scope of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Accordingly, it is to be understood that the above description is intended to be illustrative and not restrictive. Many embodiments and applications other than the examples provided would be apparent upon reading the above description. The scope should be determined, not with reference to the above description or Abstract below, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

1. A method for building a pedigree for a disease based on a family tree, the method comprising the steps of:
receiving, by a pedigree service, a family health condition request;
transmitting, by the pedigree service, a request for the family tree;
transmitting, by the pedigree service, a data request for health conditions of relatives on the family tree to an access control system;
verifying, by the access control system, consent of the relatives on the family tree;
receiving, by the pedigree service, health conditions of the relatives from a health data storage; and
drawing, by the pedigree service, a pedigree for the disease based on the health conditions of the relatives.

2. The method for building a pedigree for a disease based on a family tree of claim 1, the method further comprising:
transmitting, by the pedigree service, the pedigree to a patient.

3. The method for building a pedigree for a disease based on a family tree of claim 1, the method further comprising:
determining, by the pedigree service, which of the relatives on the family tree are affected by the disease.

4. The method for building a pedigree for a disease based on a family tree of claim 1, the method further comprising:
identifying, by the pedigree service, a pattern of inheritance.

5. The method for building a pedigree for a disease based on a family tree of claim 1, the method further comprising:
identifying, by the pedigree service, a risk for the relatives on the family tree for the disease.

6. The method for building a pedigree for a disease based on a family tree of claim 1, wherein the access control system verifies the relatives on the family tree and a signature from a service which provides the family tree.

7. The method for building a pedigree for a disease based on a family tree of claim 6, wherein the relatives on the family tree, using a Merkle tree, attach data on a blockchain with a hash value.

8. The method for building a pedigree for a disease based on a family tree of claim 7, wherein when the pedigree service requests the health condition data from the access control system, the pedigree service attaches a link to the blockchain.

9. The method for building a pedigree for a disease based on a family tree of claim 8, wherein the access control system verifies the blockchain and transmits the health condition data.

10. The method for building a pedigree for a disease based on a family tree of claim 1, wherein genome data from the patient is sent to a network which determines whether the genome data belongs to relatives on the network.

11. A pedigree service for building a pedigree for a disease based on a family tree, the pedigree service comprising:
a memory; and
a processor configured to:
receive a family health condition request;
transmit a request for the family tree;
transmit a data request for health conditions of relatives on the family tree to an access control system;
receive verification of consent for the relatives on the family tree;
receive health conditions of the relatives from a health data storage; and
draw a pedigree for the disease based on the health conditions of the relatives.

12. The pedigree service for building a pedigree for a disease based on a family tree of claim 11, the processor further configured to:
transmit the pedigree to a patient.

13. The pedigree service for building a pedigree for a disease based on a family tree of claim 11, the processor further configured to:
determine which of the relatives on the family tree are affected by the disease.

14. The pedigree service for building a pedigree for a disease based on a family tree of claim 11, the processor further configured to:
identify a pattern of inheritance.

15. The pedigree service for building a pedigree for a disease based on a family tree of claim 11, the processor further configured to:
identify a risk for the relatives on the family tree for the disease.

16. The pedigree service for building a pedigree for a disease based on a family tree of claim 11, wherein an access control system verifies the relatives on the family tree and a signature from a service which provides the family tree.

17. The pedigree service for building a pedigree for a disease based on a family tree of claim 16, wherein the relatives on the family tree, using a Merkle tree, attach data on a blockchain with a hash value.

18. The pedigree service for building a pedigree for a disease based on a family tree of claim 17, wherein when the pedigree service requests the health condition data from the access control system, the pedigree service attaches a link to the blockchain.

19. The pedigree service for building a pedigree for a disease based on a family tree of claim 18, wherein the access control system verifies the blockchain and transmits the health condition data.

20. The pedigree service for building a pedigree for a disease based on a family tree of claim 11, wherein genome data from the patient is sent to a network which determines whether the genome data belongs to relatives on the network.
